(19) United States
(12) Patent Application Publication
(10) Pub. No.: US 2022/0330862 A1
(43) Pub. Date: Oct. 20, 2022

(54) A SYSTEM FOR ADMINISTERING A FIELD SOBRIETY TEST

(71) Applicant: IMPIRICA INC., Edmonton (CA)

(72) Inventors: Aaron GRANLEY, Edmonton (CA); Peter-John BARCLAY, Edmonton (CA); Trevor GERMAIN, Edmonton (CA); Sam JENKINS, Edmonton (CA); Lejjy GAFOUR, Edmonton (CA); Darcy CHAN, Edmonton (CA); Ian PHILLIPCHUCK, Edmonton (CA)

(21) Appl. No.: 17/640,101

(22) PCT Filed: Aug. 28, 2020
(86) PCT No.: PCT/CA2020/051182

§ 371 Date: Mar. 3, 2022

(30) Foreign Application Priority Data
Sep. 5, 2019 (CA) 3 054 485

(51) Int. Cl. A61B 5/16; A61B 3/113; G16H 40/67; A61B 5/00

(52) U.S. Cl. CPC A61B 5/16; A61B 3/113; G16H 40/67; A61B 5/4863

(57) ABSTRACT

A system for administering a field sobriety test. A central server is communicable with at least one hand held device such that information entered into the at least one hand held device is communicated to the central server. The information is stored in the central server for recall. The at least one hand held device has a screen and an input. The at least one hand held device provides a step by step checklist and at least one action test. The step by step checklist is electronically interactive utilizing the input of the at least one hand held device and has at least one validation step to confirm the correct step by step procedure is followed. The at least one validation step requires that a user enter information prior to moving to a next step. The at least one action test has at least two common occurrence buttons. The at least two common occurrence buttons are electronically interactive utilizing the input of the at least one hand held device.

### FIELD OF THE DISCLOSURE

The present application relates generally to a system for administering a field sobriety test.

### BACKGROUND

This section provides background information to facilitate a better understanding of the various aspects of the invention. It should be understood that the statements in this section of this document are to be read in this light, and not as admissions of prior art.

Emergency responders are often required to provide sobriety tests while in the field. The standard field sobriety test is standardized through the International Association Chiefs of Police. When performing a standard field sobriety test, test administrators move through a series of steps and questions while both watching the person being tested and writing notes for their records. It is common for the administrators to take their eyes off of the person being tested to write their notes. This can lead to potential missed signs of impairment. It is also possible that the test administrator misses a step or question that could negatively impact their ability to charge a party if there is an apparent impairment.

### BRIEF SUMMARY

There is provided a system for administering a field sobriety test. A central server is communicable with at least one hand held device such that information entered into the at least one hand held device is communicated to the central servicer. The information is stored in the central server for recall. Recall may be made to one of the hand held devices or a different device such as a laptop, desktop computer or printer. The at least one hand held device has a screen and an input. The at least one hand held device provides a step by step checklist and at least one action test. The step by step checklist is electronically interactive utilizing the input of the at least one hand held device. The step by step checklist has at least one validation step to confirm the correct step by step procedure is followed. The at least one validation step requires that a user enter information prior to moving to a next step. The at least one action test has at least two common occurrence buttons. The at least two common occurrence buttons are electronically interactive utilizing the input of the at least one hand held device.

In one embodiment, the step by step checklist includes time records, selections for weather conditions, lighting, road surface conditions and location. Location may be determined by GPS.

In one embodiment, the step by step checklist includes a correction feedback loop allowing the user to confirm that the data entered is correct or allow the user to correct the data.

In one embodiment, the at least one action test includes a horizontal gaze nystagmus test. The horizontal gaze nystagmus test have at least one affirmative feedback loop such that a negative response does not allow the user to proceed until a positive response is entered.

In one embodiment, the negative and positive responses entered in the horizontal gaze nystagmus test are time stamped.

In one embodiment, the at least one action test includes a test correction feedback loop allowing the user to confirm the data entered is correct or allowing the user to correct the data.

In one embodiment, the at least one action test is a walk and turn test. In the walk and turn test, the common occurrence buttons include a step counter and at least one impairment indicators. Impairment indicators may include losses of balance, early starts raising of the arms, missing a heel to toe step, not taking the correct number of steps, performing an incorrect turn or not maintaining the walk. A timer may also be included.

In another embodiment, the at least one action test is a one leg stand test. In the one leg stand test, the common occurrence buttons include at least one impairment indicators and an arm movement indicator. Impairment indicators may include hopping, excessive leaning of the body, touching the ground with a raised foot or a slow or fast internal clock count. A timer may also be included.

In one embodiment, the central server further comprises a transcript generator module for generating documents in a predetermined format based upon the information being stored in the central server.

In one embodiment, decision points are calculated during each of the at least one action tests. The decision points are calculated based upon the interaction with the at least two common occurrence buttons and are indicative of impairment.

### BRIEF DESCRIPTION OF THE DRAWINGS

These and other features will become more apparent from the following description in which references are made to the following drawings, in which numerical references denote like parts. The drawings are for the purpose of illustration only and are not intended to in any way limit the scope of the invention to the particular embodiments shown.

FIG. 1 is a schematic view of the system for administering a field sobriety test.

FIG. 2 is a diagram of an authentication page of the system.

FIG. 3 is a diagram of a case management page of the system.

FIG. 4 is a diagram of a first page of a step by step checklist.

FIG. 5 is a diagram of a second page of the step by step checklist shown in FIG. 4.

FIG. 6 is a diagram of a horizontal gaze nystagmus test.

FIG. 7 is a diagram of a correction feedback loop for the horizontal gaze nystagmus test.

FIG. 8 is a diagram of a first page of a walk and turn test.

FIG. 9 is a diagram of a second page of the walk and turn test.

FIG. 10 is a diagram of the second page of the walk and turn test after the subject turns.

FIG. 11 is a diagram of the test correction feedback loop of the walk and turn test.

FIG. 12 is a diagram of a results page of the walk and turn test.

FIG. 13 is a diagram of a correction feedback loop for the walk and turn test.

FIG. 14 is a diagram of a first page of a one leg stand test.

FIG. 15 is a diagram of a second page of the one leg stand test.

FIG. 16 is a diagram of a summary of the one leg stand test.

FIG. 17 is a diagram of a report page of the one leg stand test.

FIG. 18 is a diagram of a correction feedback loop for the one leg stand test.

FIG. 19 is a diagram of a summary test page.

### DETAILED DESCRIPTION OF THE PREFERRED EMBODIMENTS

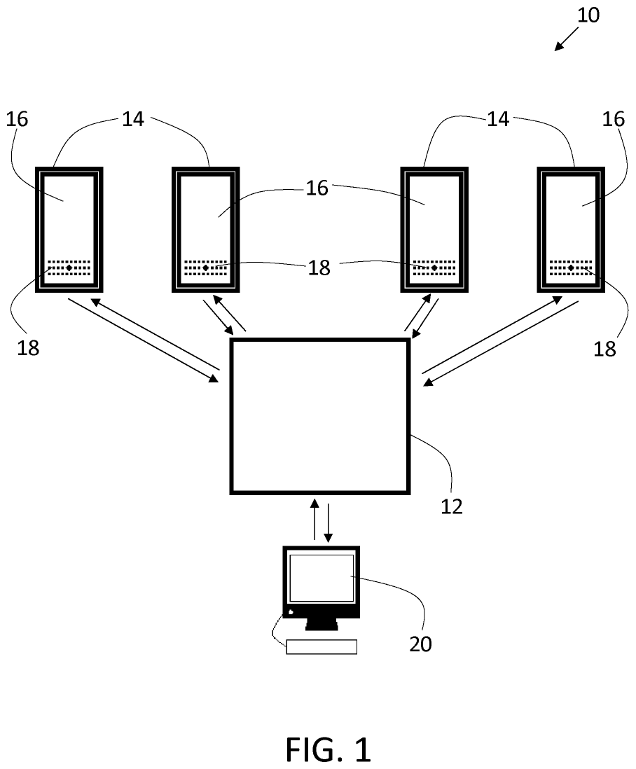

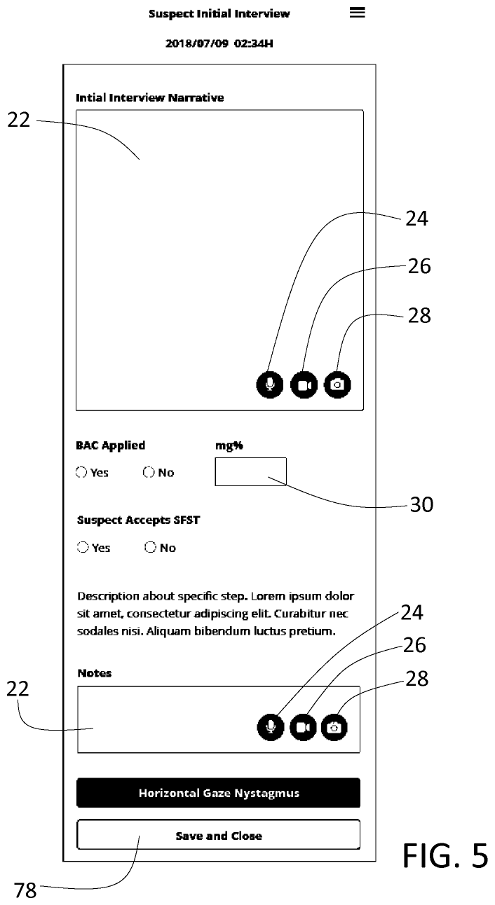

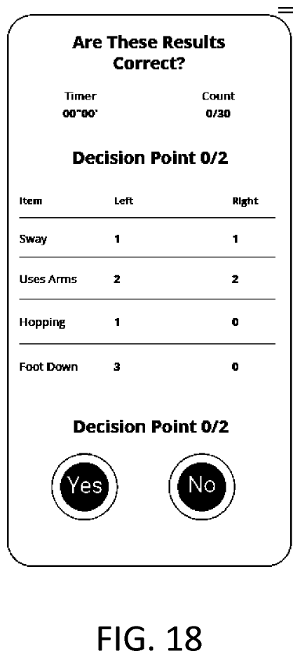

A system for administering a field sobriety test, generally identified by reference numeral 10, will now be described with reference to FIG. 1 through FIG. 19.

Referring to FIG. 1, a system for administering a field sobriety test 10 includes a central server 12 and at least one hand held device 14. Central server 12 is communicable with hand held devices 14 such that information entered into hand held devices 14 is communicated to central server 12. The information is stored in central server 12 for recall by a hand held device 14 or other device 20 such as a laptop, desktop computer or printer. This allows information entered into hand held devices 14 to be accessible from other locations by other devices. Hand held devices 14 have a screen 16 and an input 18. Input 18 may include a keyboard, mouse, touch screen or any other type of input known to a person skilled in the art. In the embodiment shown, hand held device 14 is a smart cell phone with a camera, microphone and wireless connections.

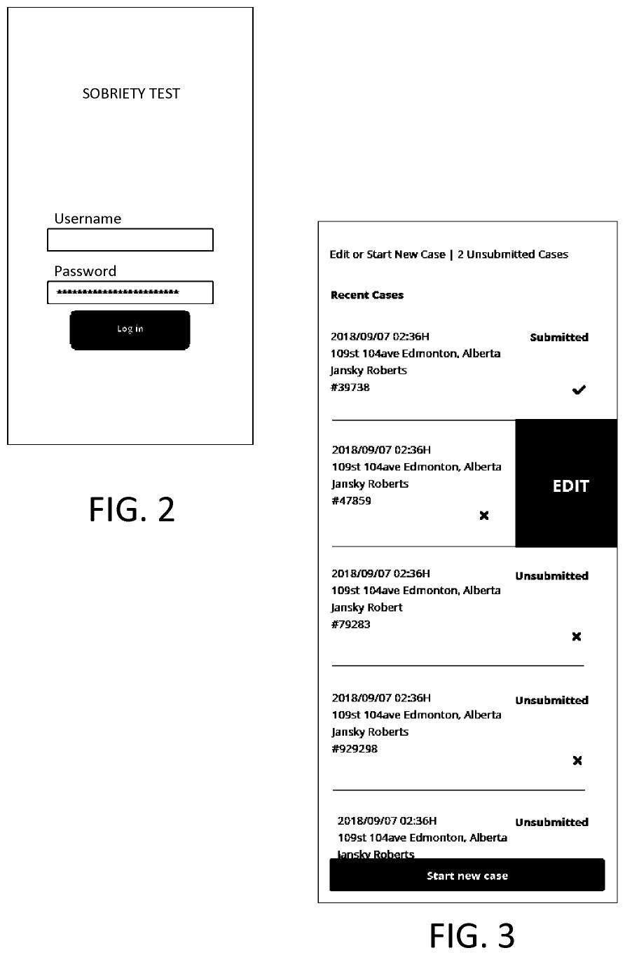

Referring to FIG. 2, an authentication protocol requiring a username and password may be provided to limit access to system 10. This allows authorized personnel to view test results or administer tests while limiting the ability of non-authorized parties from accessing the information. This can become important when information entered into system 10 is confidential in nature.

Hand held devices 14 provide a step by step checklist that is electronically interactive by utilizing input 18 of hand held devices 14. Step by step checklist has at least one validation step to confirm that the correct step by step procedure is followed. The at least one validation step requires that a user enter information prior to moving to a next step. In the embodiment shown in FIG. 5, step by step checklist includes time records, selections for weather conditions, selections for lighting, selections for road surface conditions and a location. This allows a test administrator to quickly choose the appropriate options and proceed with the sobriety test. In the embodiment shown, a blank fillable space 22 is provided to allow a test administrator to quickly include notes when relevant conditions are not described by the selections available. This may include the amount of traffic, sudden and unexpected changes in weather or any other note that the test administrator believes to be relevant. The test administer may record audio by activating an audio button 24, record video by activating a video button 26 or take a photograph by activating a camera button 28. In the embodiment shown, location is determined by GPS that is either connectible to hand held device 14 or is included as a part of hand held device 14. If GPS is unavailable, a test administrator may manually input a location.

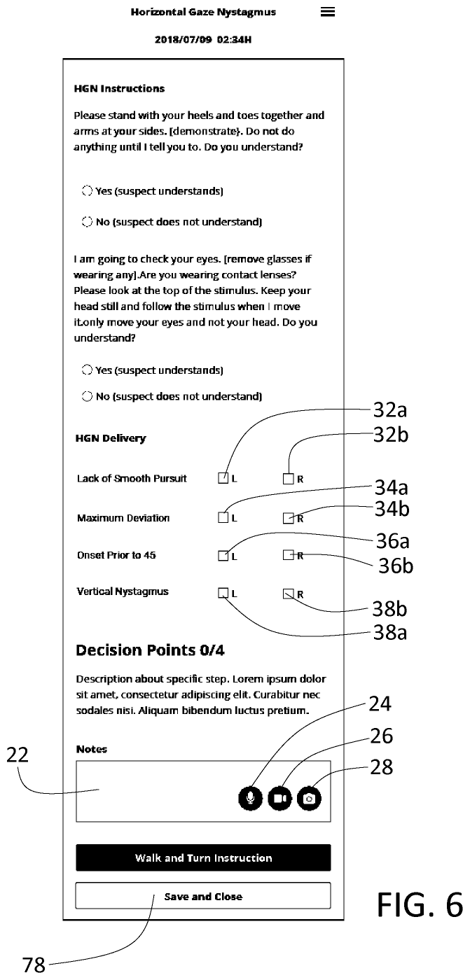

Referring to FIG. 6, once general relevant conditions are entered, the step by step checklist provides for the option to report on whether or not a blood-alcohol concentration (BAC) has been applied. This is generally completed through a breathalyzer. The step by step checklist allows the test administrator to confirm whether a BAC was applied and, if it was, provides a blank space 30 to record the BAC reading. In the embodiment shown, a blank fillable space 22 is provided to allow a test administrator to include notes from the initial interview that the test administrator believes to be relevant. The test administer may record audio by activating an audio button 24, record video by activating a video button 26 or take a photograph by activating a camera button 28. Following completion of this step, the step by step checklist is completed and the test administrator moves on to the at least one action tests. It will be understood by a person skilled in the art that different questions, options and observations may be included within the step by step checklist.

Action tests are provided for assisting in determining the sobriety of a subject. Action tests have at least two common occurrence buttons. The common occurrence buttons are electronically interactive by utilizing the input of the hand held devices 14. In the embodiments shown, common occurrence buttons may be positioned equidistant around a central location. This creates a joystick functionality that allows for easy directional input of impairment indicators. As test administrators become more accustomed to utilizing the action tests, they may be able to spend more time watching the test subject while still accurately inputting information with the input of the hand held device. In the embodiments shown, action tests include a horizontal gaze nystagmus test, a walk and turn test and a one leg stand test. It will be understood by a person skilled in the art that other action tests could be included.

Referring to FIG. 6, the horizontal gaze nystagmus test begins with the test administrator providing instructions to the subject. In the embodiment shown, two affirmative feedback loops are provided that require that the test administrator enter a “YES” response prior to moving forward to the next step. If a subject indicates “NO”, a time stamp of the “NO” response is made, however a test administrator cannot proceed until a “YES” response is received from the subject. In the embodiment shown, the next step button for is greyed out and unavailable until a “YES” response is entered by the test administrator. It will be understood by a person skilled in the art that a single affirmative feedback loop or multiple affirmative feedback loops could be used depending upon how the horizontal gaze nystagmus test was set up. In the embodiment shown, the first affirmative feedback loop relates to the test administrator describing and demonstrating the correct posture for the test and requiring an affirmative response from the subject regarding their understanding. The second affirmative feedback loop relates to the test administrator indicating what the test is for and providing instructions to the subject. Prior to starting the test, an affirmative response from the subject regarding their understanding is required. The common occurrence buttons sets out the reactions that are looked for during a horizontal gaze nystagmus test. In the embodiment shown, a lack of smooth pursuit 32a and 32b, maximum deviation 34a and 34b, onset prior to 45 degrees 36a and 36b and vertical nystagmus 38a and 38b are looked for in relation to both the left and right eye of the subject are provided as common occurrence buttons. The occurrence of these reactions are input into hand held device 14.

Based upon the information input into hand held device 14, decision points are calculated. Decision points are determined based upon the presence of clues that show up during the horizontal gaze nystagmus test. The decision points may be indicative of impairment. In the horizontal gaze nystagmus test, decision points are determined by the positive observation of a lack of smooth pursuit, maximum deviation and onset prior to 45 degrees in each eye. Vertical gaze nystagmus may be used an additional impairment indicator as it may show severe impairment or neurological issues. In the present embodiment, vertical gaze nystagmus is not used in calculating decision points, however it will be understood by a person skilled in the art that it could be included. In the embodiment shown, a score out of four is shown, however it will be understood by a person skilled in the art that a different scoring matrix could be used. It is possible to receive a score as high as 6/4 if a positive observation is made for each of the four reactions provided in both eyes. If a subject receives a score of 4/4 or greater, it may be indicative of impairment. A score of 3/4 or lower indicates that for that specific test impairment was not indicated. A test administrator may still find impairment when viewing the horizontal gaze nystagmus test results with the rest of the field sobriety test. It will be understood by a person skilled in the art that decision points may be determined by other means including algorithms or artificial intelligence. In the embodiment shown, a blank fillable space 22 is provided to allow a test administrator to include notes. This may include the test administrators observations of the subject while providing instructions or performing the horizontal gaze nystagmus test. The test administer may record audio by activating an audio button 24, record video by activating a video button 26 or take a photograph by activating a camera button 28.

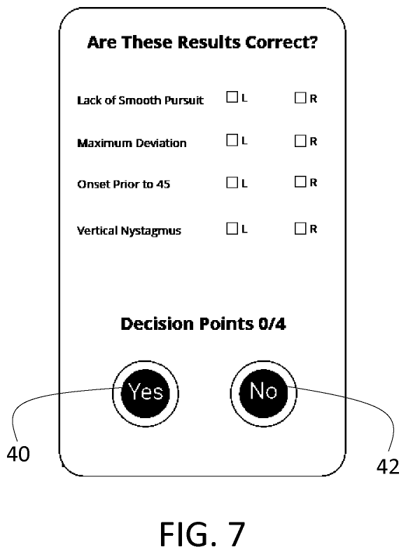

Referring to FIG. 7, upon completion of the horizontal gaze nystagmus test, a test administrator is provided with a correction feedback loop to confirm that the information entered regarding the horizontal gaze nystagmus test is correct. If it is, the test administrator can confirm the information by activating the YES button 40 and move forward with sobriety test. If it is not, the test administrator activates the NO button 42 and is returned to the horizontal gaze nystagmus test for correction before proceeding.

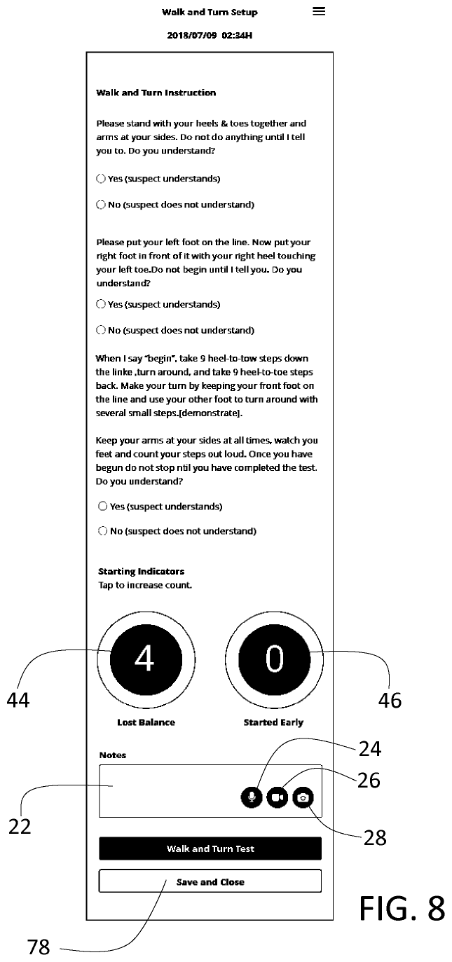

Referring to FIG. 8, the walk and turn test begins with the test administrator providing instructions to the subject. In the embodiment shown, three affirmative feedback loops are provided that require that the test administrator enter a “YES” response prior to moving forward to the next step. If a subject indicates “NO”, a time stamp of the “NO” response is made, however a test administrator cannot proceed until a “YES” response is received from the subject. In the embodiment shown, the next step button performing the walk and turn test is greyed out and unavailable until three “YES” responses are entered by the test administrator. It will be understood by a person skilled in the art that a single affirmative feedback loop or multiple affirmative feedback loops could be used depending upon how action test was set up. In the embodiment shown, in the embodiment shown, common occurrence buttons are provided for counting the number of times a subject loses balance 44 or starts the test early 46. When ready, the test administrator proceeds to the movement portion of the walk and turn test. In the embodiment shown, a blank fillable space 22 is provided to allow a test administrator to include notes. This may include the test administrators observations of the subject while providing instructions or performing the horizontal gaze nystagmus test. The test administer may record audio by activating an audio button 24, record video by activating a video button 26 or take a photograph by activating a camera button 28.

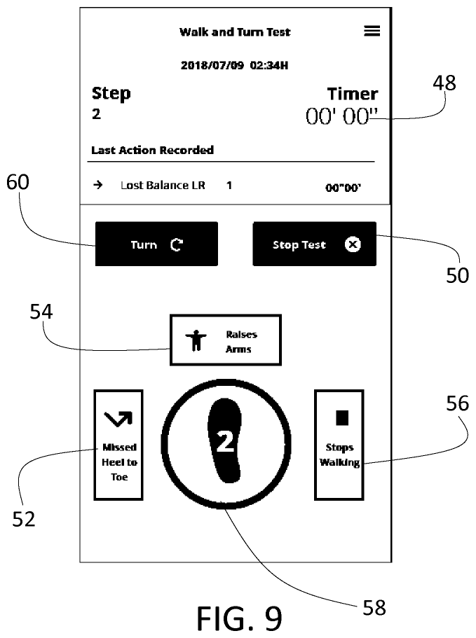

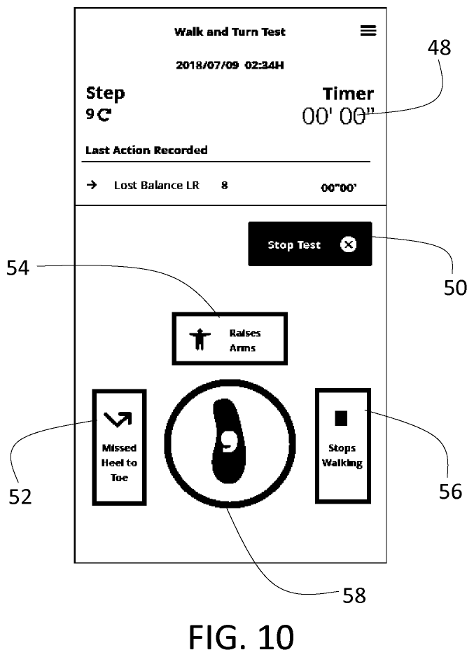

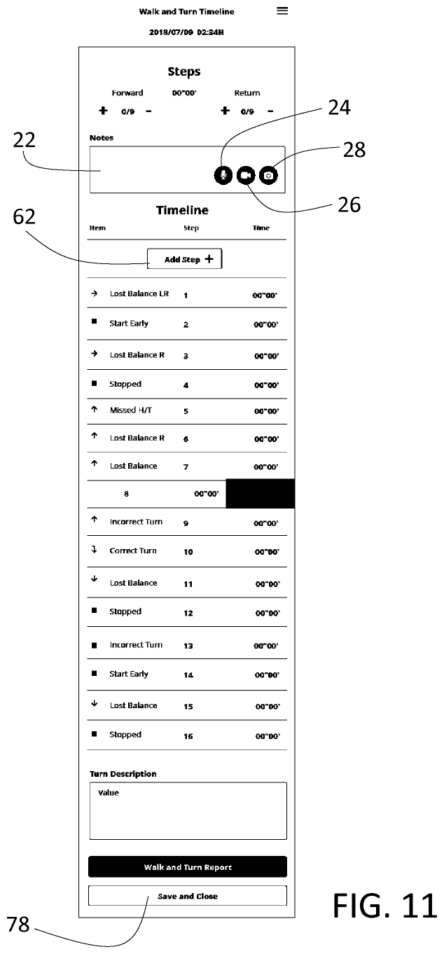

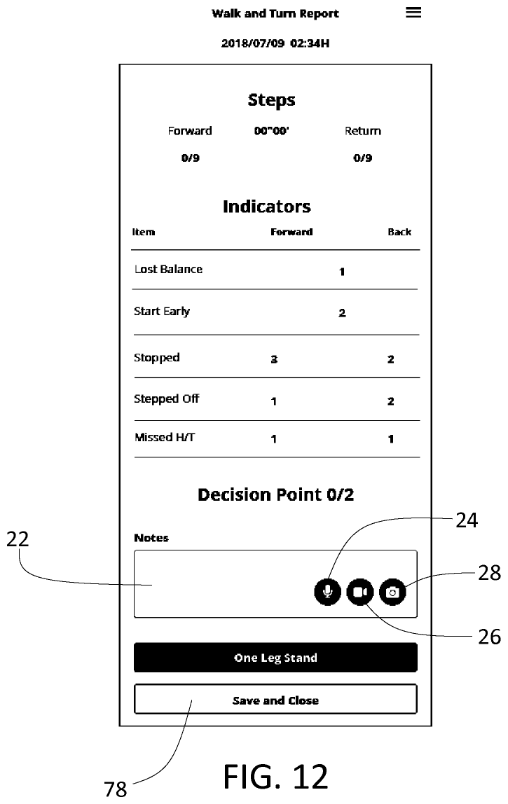

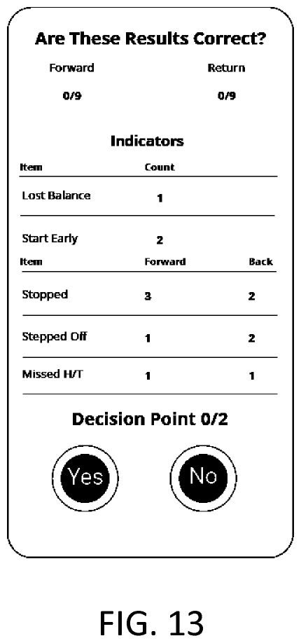

Referring to FIG. 9, the walk and turn test has a timer 48 that is started when the start test button 50 is chosen and stopped when the stop test button is chosen. In the embodiment shown, the start test button and the stop test button are the same, however it will be understood that different buttons may be used to start and stop the timer. For the walk and turn test, common occurrence buttons include a missed heel to toe step button 52, a raised arms button 54, a stops walking button 56 and a step button 58. In the embodiment shown, the buttons are arrayed such that step button 58 is located in a location central to the missed heel to toe step button 52, the raised arms button 54 and the stops walking button 56. Timer 48 may also be started by pressing step button 58. When utilizing step button 58, a test administrator may contact step button 58 and drag their finger to correspond with the direction of a misstep taken by the subject. If the subject misses a heel to toe step, raises their arms or stops walking, the appropriate button can be activated. A time stamp of the activation of a common occurrence button may be made to track activation of common occurrence buttons during the walk and turn test. A turn button 60 is provided for the test administrator to activate when the subject turns to walk back towards them. In the embodiment shown in FIG. 10, after turn button 60, shown in FIG. 9, has been activated, step button 58 may rotate to show that movement in a second direction is being tracked. As can be seen, turn button 60 may disappear after activation. It will be understood by a person skilled in the art that step button 58 is not required to rotate. Upon pressing the stop test button 50, the test administrator is provided with a summary of the walk and turn test, shown in FIG. 11 and FIG. 12. Referring to FIG. 13, the summary provides the test administrator with a test correction feedback loop that allows them to confirm the timeline and actions of the steps in the walk and turn test. Referring to FIG. 11, options are provided for adding missed steps by activating the add step button 62, deleting incorrect information by interacting with the timeline and providing notes about observations made during the test. In the embodiment shown, a blank fillable space 22 is provided to allow a test administrator to include notes. This may include the test administrators observations of the subject while providing instructions or performing the walk and turn test. The test administer may record audio by activating an audio button 24, record video by activating a video button 26 or take a photograph by activating a camera button 28. Referring to FIG. 12, based upon the information input into hand held device 14 during the walk and turn test, decision points are calculated. Decision points are determined based upon the presence of clues that show up during the walk and turn test. The decision points may be indicative of impairment. In the walk and turn test, decision points are determined by the first instance that each common occurrence button is activated or a turn is performed incorrectly. In the embodiment shown, a score out of two is shown, however it will be understood by a person skilled in the art that a different scoring matrix could be used. If a subject receives a score of 2/2 or greater, it may be indicative of impairment. A score of 1/2 or lower indicates that for that specific test impairment was not indicated. A test administrator may still find impairment when viewing the walk and turn test results with the rest of the field sobriety test. It will be understood by a person skilled in the art that decision points may be determined by other means including algorithms or artificial intelligence. In the embodiment shown, the walk and turn test utilizes two test correction feedback loops that allows a user to confirm the test results.

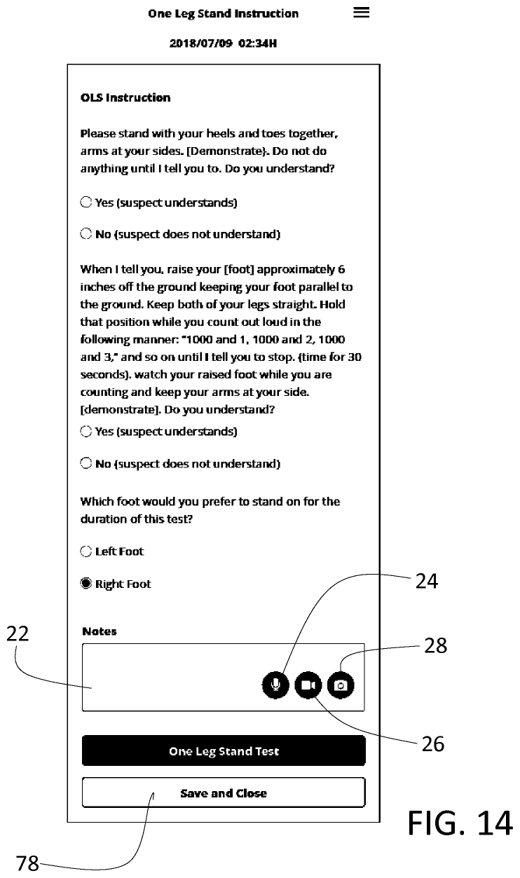

Referring to FIG. 14, the one leg stand test begins with the test administrator providing instructions to the subject. In the embodiment shown, two affirmative feedback loops are provided that require that the test administrator enter a “YES” response prior to moving forward to the next step. If a subject indicates “NO”, a time stamp of the “NO” response is made, however a test administrator cannot proceed until a “YES” response is received from the subject. In the embodiment shown, the next step button for performing the one leg stand test is greyed out and unavailable until two “YES” responses are entered by the test administrator. It will be understood by a person skilled in the art that a single affirmative feedback loop or multiple affirmative feedback loops could be used depending upon how action test was set up. The test administrator confirms with the subject which foot they prefer to stand on for the test and confirms the appropriate foot before proceeding with the test. The test administrator may also update information related to which foot is preferred after the evaluation to reflect the appropriate foot. In the embodiment shown, a blank fillable space 22 is provided to allow a test administrator to include notes. This may include the test administrators observations of the subject while providing instructions or performing the walk and turn test. The test administer may record audio by activating an audio button 24, record video by activating a video button 26 or take a photograph by activating a camera button 28. When ready, the test administrator proceeds to the execution of the one leg stand test.

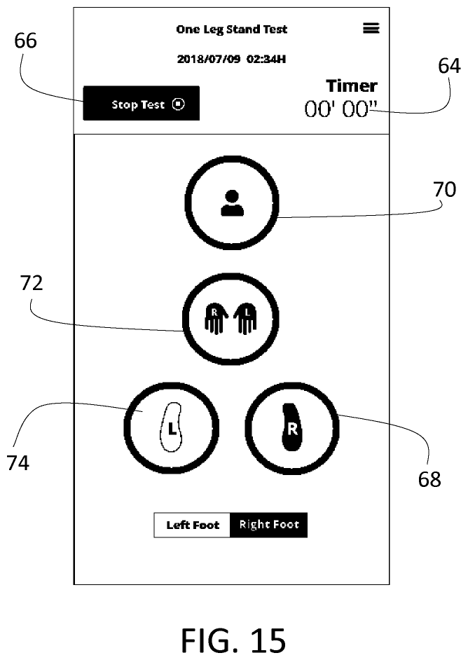

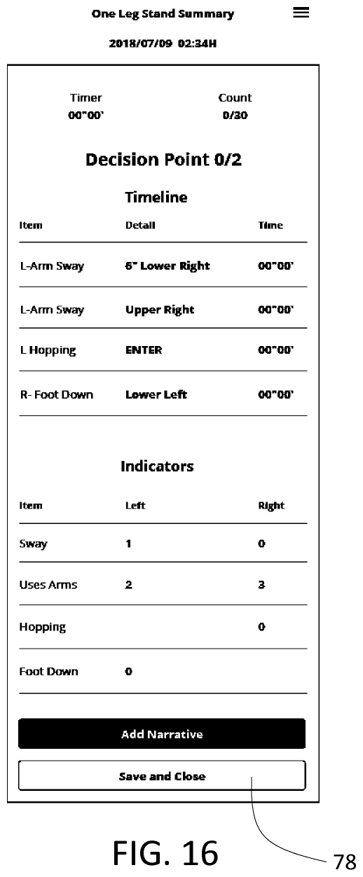

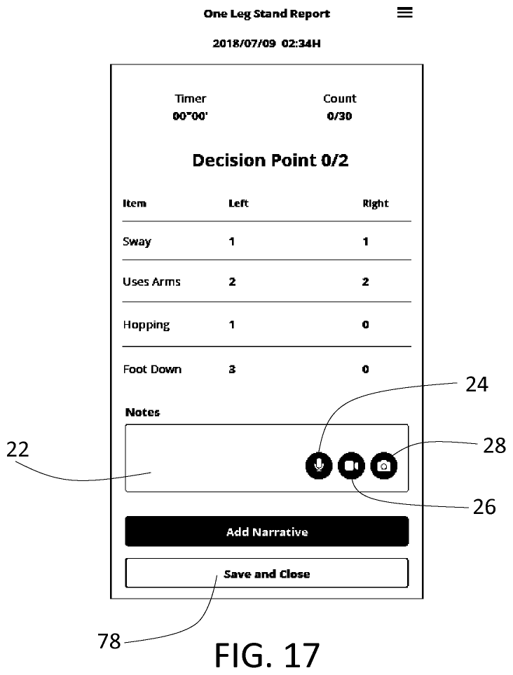

Referring to FIG. 15, the one leg stand test has a timer 64 that is started when the start test button 66 is chosen and stopped when the stop test button is chosen. In one embodiment of the one leg stand test, the test is stopped automatically after 30 seconds has passed after timer 64 is started. The stop test button can be pressed prior to the elapsing of 30 seconds if there is a reason to end the test early. Reasons to end the test may include a safety concern or any other issue occurring during the test. In the embodiment shown, the start test button and the stop test button are the same, however it will be understood that different buttons may be used to start and stop the timer. For the one leg stand test, common occurrence buttons include a hop button 68, a sway button 70, a uses arms button 72 and a foot down button 74. When utilizing the sway button 70, the uses arms button 72 and the foot down button 74, a test administrator may contact the button and drag their finger to correspond with the direction of a movement taken by the subject. If the subject hops, the appropriate button 68 can be activated. A number entry pad may be brought up to allow the test administrator to enter the subject's verbal count number at the time that the hop occurred. Upon pressing the stop test button 66, the test administrator is provided with a summary of the one leg stand test, shown in FIG. 16. Referring to FIG. 17, the summary provides the test administrator with a test correction feedback loop that allows them to confirm the timeline and actions of the one leg stand test. Options are provided for adding missed events and providing notes about observations made during the test. These notes may also include the verbal count number that the subject being tested reached at the end of the timed test. In the embodiment shown, a blank fillable space 22 is provided to allow a test administrator to include notes. This may include the test administrators observations of the subject while providing instructions or performing the walk and turn test. The test administer may record audio by activating an audio button 24, record video by activating a video button 26 or take a photograph by activating a camera button 28. Based upon the information input into hand held device 14 during the one leg stand test, decision points are calculated. Decision points are determined based upon the presence of clues that show up during the one leg stand test. The decision points may be indicative of impairment. In the one leg stand test, decision points are determined by the first instance that each common occurrence button is activated. In the embodiment shown, a score out of two is shown, however it will be understood by a person skilled in the art that a different scoring matrix could be used. If a subject receives a score of 2/2 or greater, it may be indicative of impairment. A score of 1/2 or lower indicates that for that specific test impairment was not indicated. A test administrator may still find impairment when viewing the one leg stand test results with the rest of the field sobriety test. A further impairment indication may include a fast or slow internal clock count. An internal clock count is determined by the number reached by a test subject during the measured time of the test. For example, a test subject's internal clock may be fast if they have counted to 45 in a 30 second test. It will be understood by a person skilled in the art that decision points may be determined by other means including algorithms or artificial intelligence. In the embodiment shown, the one leg stand test utilizes two test correction feedback loops that allows a user to confirm the test results.

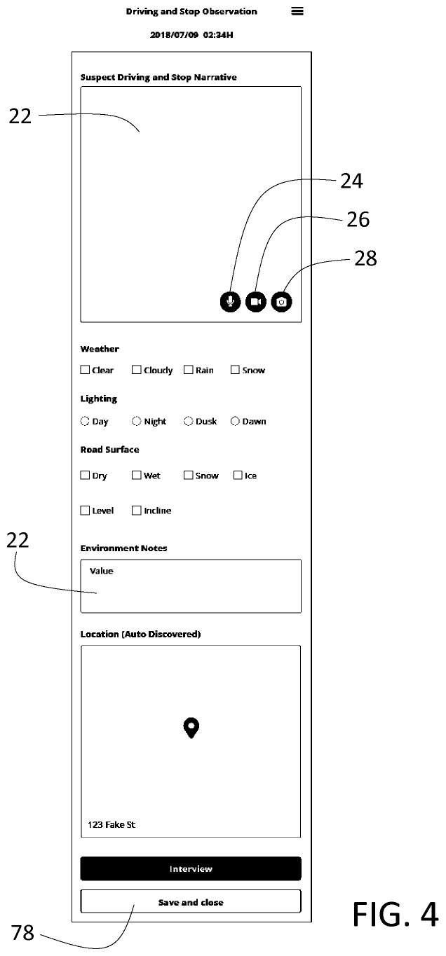

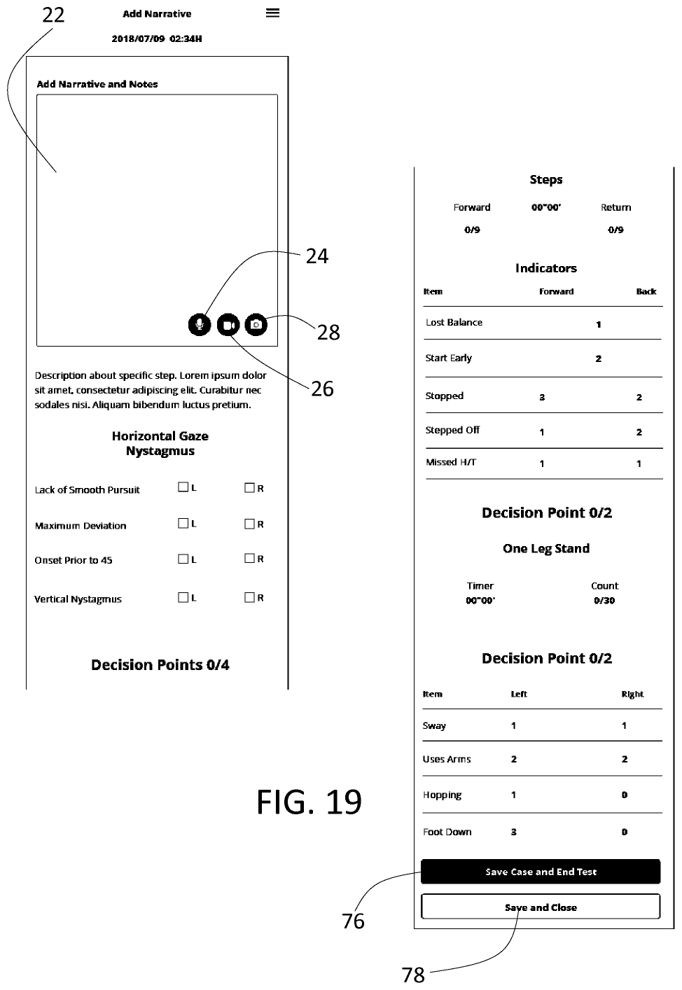

Referring to FIG. 19, following the completion of the at least one action test, the test administrator is provided with an overall summary of the test results from the at least one action tests. The decision points for each test are also shown. In the embodiment shown, a blank fillable space 22 is provided to allow a test administrator to include notes. This may include the test administrators observations related to the sobriety test. The test administer may record audio by activating an audio button 24, record video by activating a video button 26 or take a photograph by activating a camera button 28. A test administrator can complete the test, save the case and send the case to the central server by activating the end test button 76. During the step by step checklist and the action tests, the information entered into the system is saved and communicated to the central server. It is possible for uncompleted tests to be submitted to the central server. During the step by step checklist and the action tests, it is also possible to stop the test for a number of reasons. In the embodiment shown, when stopping a test, a save and close button 78 may be activated. The test administrator may be prompted to provide notes as to why the test has been stopped, may allow them to pause the test and return to a case list. Referring to FIG. 3, a case management page is provided that lists the tests that have been performed, their status and provides for the ability to edit or un-pause the tests.

Central server 12 may have a transcript generator module that allows for documents to be generated in a predetermined format based upon the information that is stored in the central server and collected using the hand held devices 14. This can allow for simple completion of paperwork in an orderly format. This can help to avoid transcription errors and improve the speed at which documents created and files completed.

Any use herein of any terms describing an interaction between elements is not meant to limit the interaction to direct interaction between the subject elements, and may also include indirect interaction between the elements such as through secondary or intermediary structure unless specifically stated otherwise.

In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be one and only one of the elements.

It will be apparent that changes may be made to the illustrative embodiments, while falling within the scope of the invention. As such, the scope of the following claims should not be limited by the preferred embodiments set forth in the examples and drawings described above, but should be given the broadest interpretation consistent with the description as a whole.

What is claimed is:

1. A system for administering a field sobriety test, comprising:
a central server communicable with at least one hand held device such that information entered into the at least one hand held device is communicated to the central server, the information being stored in the central server for recall;
the at least one hand held device having a screen and an input, the at least one hand held device providing:
a step by step checklist being electronically interactive utilizing the input of the at least one hand held device and having at least one validation step to confirm the correct step by step procedure is followed, the at least one validation step requiring that a user enter information prior to moving to a next step;
at least one action test having at least two common occurrence buttons, the at least two common occurrence buttons being electronically interactive utilizing the input of the at least one hand held device.

2. The system of claim 1 wherein the step by step checklist includes time records, selections for weather conditions, lighting, road surface conditions and location.

3. The system of claim 2 further including a GPS for determining location.

4. The system of claim 1 wherein the step by step checklist includes a correction feedback loop allowing the user to confirm that the data entered is correct or allowing the user to go back to correct the data.

5. The system of claim 1 wherein the at least one action test having at least one affirmative feedback loop such that a negative response does not allow the user to proceed until a positive response is entered.

6. The system of claim 5 wherein the negative response and the positive response are time stamped.

7. The system of claim 1 wherein the at least one action test includes a test correction feedback loop allowing the user to confirm the data entered is correct or allowing the user to correct the data.

8. The system of claim 1 wherein the at least one action test includes a horizontal gaze nystagmus test.

9. The system of claim 7 wherein the common occurrence buttons of the horizontal gaze nystagmus test include a lack of smooth pursuit, maximum deviation, onset prior to 45 degrees and vertical nystagmus.

10. The system of claim 1 wherein the at least one action test is a walk and turn test.

11. The system of claim 10 wherein the common occurrence buttons of the walk and turn test include a step counter and at least one impairment indicator.

12. The system of claim 10 wherein the walk and turn test further comprises a timer.

13. The system of claim 1 where the at least one action test is a one leg stand test.

14. The system of claim 13 wherein the common occurrence buttons of the one leg stand test include at least one impairment indicator and an arm movement indicator.

15. The system of claim 13 wherein the one leg stand test further comprises a timer.

16. The system of claim 1 wherein central server further comprises a transcript generator module for generating documents in a predetermined format based upon the information being stored in the central server.

17. The system of claim 1 wherein decision points are calculated during each of the at least one action tests, the decision points being calculated based upon the interaction with the at least two common occurrence buttons and being indicative of impairment.
